(19) United States
(12) Patent Application Publication
(10) Pub. No.: US 2012/0220752 A1
(43) Pub. Date: Aug. 30, 2012

(54) TRI-AMINO RELEASING FULVATE

(76) Inventor: STEVEN R. SCHUTT, Prescott, AZ (US)

(21) Appl. No.: 13/285,151

(22) Filed: Oct. 31, 2011

Related U.S. Application Data
(60) Provisional application No. 61/447,558, filed on Feb. 28, 2011.

(51) Int. Cl. C07K 5/02

(52) U.S. Cl. 530/317

(57) ABSTRACT

A free amino acid and/or amino ion-releasing molecule useful for a wide variety of medical and cosmetic applications. The chemical name of the new molecule nino-gl ino-parabenzoate, sometimes referred to herein as TAFA. Its chemical structure is graphically depicted according to structure VIII

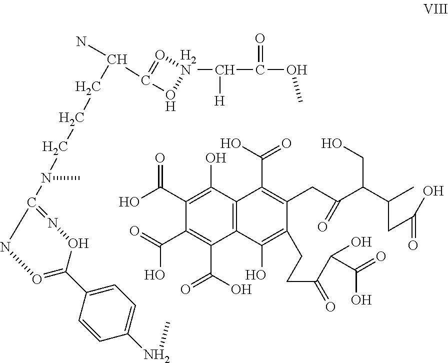

### FIELD OF THE INVENTION

The present invention claims priority of provisional application 61447558, the disclosure of which is herein incorporated by reference in its entirety.

The present invention generally relates to amino acids and compounds and complexes made therewith. Many compounds and complexes formed from and between amino acids present significant hydrogen bonding interactions, both internally as well as with the surrounding solvent system.

### BACKGROUND OF THE INVENTION

Amino acids are well known in the art and are taught In most high school curriculums to be an organic compound containing an amine group (—NH2) which is typically adjacent (or α) to the acid or carboxylic acid (—COON) moiety. The simplest amino acid is alanine abbreviated as Ala or A and has the graphical representation shown in Structure I.

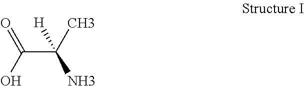

It is common practice in the art when drawing graphical representations of molecules especially organic compounds, to assume a carbon at each vertex, unless another atom is shown, and to assume the presence of the number of hydrogens needed to complete the carbons octet, which is four bonds. Following this guideline, alanine would be drawn as in Structure II.

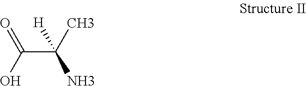

Arginine is an amino acid and is abbreviated ARG or R and is depicted graphically as Structure

III. Arginine is produced In the human body through various metabolic pathways including the digestion of proteins. Arginine is also known as 2-amino-S-guanidinovaleric acid yet, according to the International Union of Pure and Applied Chemistry naming system should be named as the amino acid preferably. Arginine is commercially available from numerous sources, including Sigma Aldrich of St. Louis Mo. USA.

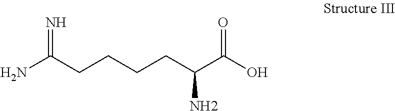

PABA is an acronym representing Para-Amino Benzoic Acid and is depicted graphically In Structure IV. PABA IS typically a white free-flowing solid available from numerous commercial sources, such as Sigma-Aldrich of St. Louis, Mo., USA. PABA is known to provide several health benefits, is a powerful uv absorber and free-radical scavenger. Owing to the para orientation between the carboxylic acid moiety and the amino group, PABA is a strong hydrogen bonding partner and often found in ionically bonded interactions.

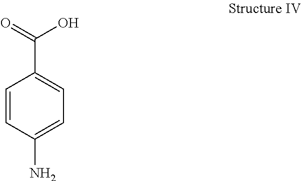

In 1995 applicant filed a patent application describing a compound called arginine aminobenzoate made by combining arginine with PABA. U.S. Pat. Nos. 5,734,080, 6,365,167 and 6,585,988 were issued based on disclosures relating to the 1995 original application. Arginine benzoate was made by combining PABA in isopropanol at 60° C. with agitation, to which was added the slurry made from the addition of arginine (either 1 or d or a mixture thereof) to isoproponal also at 60° C. The resultant mixture was filtered to yield the desired reaction product.

Glycine is the simplest amino acid, is the only one absent a chiral center adjacent the amine and is shown graphically as Structure V.

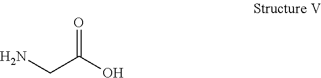

According to IUPAC nomenclature, glycine is also known as amino-acetic acid and is available as a dietary supplement from numerous commercial sources, including Sigma Aldrich of St. Louis, Mo., USA. As a dietary supplement, glycine is reported to provide numerous health benefits including antacid treatment, general antibiotic and gastric and bladder lavage agent.

Carbohydrate derived Fulvic Acid (hereinafter “CHDFA”) has the general molecular structure depicted in Figure VII with C₂₇H₂₆O₁₈ as the molecular formula, including 18 hydrogen bond acceptors and 10 hydrogen bond donors. CHDFA is produced through the wet oxidation of refined sucrose, using medical grade oxygen and water purified through reverse osmosis.

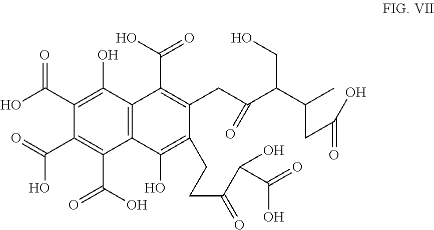

CDFA has several highly polar and polarizable functional groups, providing ample sites for powerful hydrogen bond and ionic interactions. Perhaps through these bonding sites, CHDFA is known to be a powerful chelating and ionic binding agent. CHDFA has been shown capable of transporting many beneficial amino acids and essential minerals across skin and other membranes. Through these powerful ionic interactions CHDFA forms a stable complex with the amino acids para-Amino benzoic acid, arginine, glycine and alanine to forming a complex represented by Figure VIII

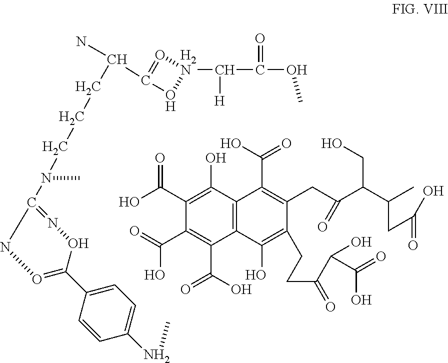

What is needed is a better product for providing free amino ions in the presence of a strong chelating complex for topical, oral and internal delivery.

### SUMMARY OF THE INVENTION

The present invention provides a free amino ion releasing molecule useful for a variety of medical, cosmetic and cosmeceutical applications. The new complex or molecule according to the present invention is arginino-glycino-parabenzoate Fulvate, according to IUPAC naming rules. Although the exact molecular orientation is a challenge to determine, one reasonable graphical representation is In Structure VII where the amine in PABA is hydrogen bonded with the acid portion of glycine and the amine portion from arginine is hydrogen bonded with the acid portion of the PABA. In this representation, the nitrogen's are drawn without hydrogen's to more clearly demonstrate the iconic interactions.

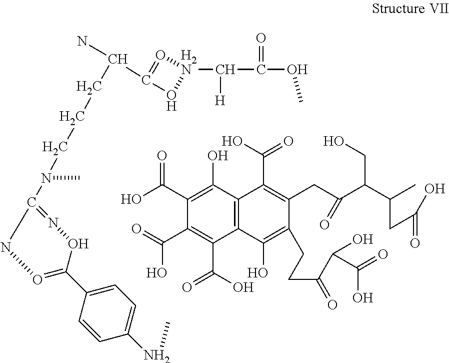

The new amino acid releasing complex can be formed according to either of two methods. The first method includes the steps of mixing arginine and glycine in water at about room temperature, to provide the glycine-arginate salt intermediate as expected according to traditional acid-base chemical understanding. The intermediate IS then combined with PABA and CHDFA to yield the final composition. The second method inverts the order whereby PABA and CHDFA are combined with glycine to form an intermediate para-aminobenzo-glycin-fulvate, according to IUPAC naming guidelines. This intermediate is then combined with arginine to provide the desired product, arginino-glycino-parabenzene-fulvate, according to IUPAC naming rules, but which shall be referred to as “tri-amino fulvate” and/or “TAFA” In this disclosure and is graphically represented as Structure VIII. Of course the individual molecules are able to adopt a more energy beneficial orientation, perhaps influenced by temperature or other ionic interactions yet maintaining the essential molecular formula of one PABA, one arginine one glycine and one CHDFA as further depicted in Structure VIII.

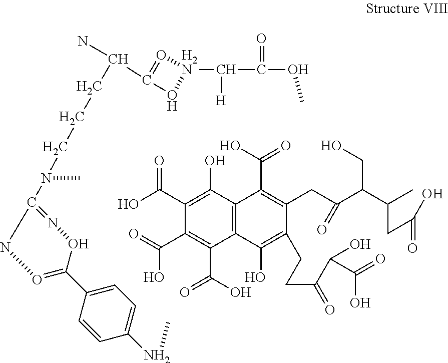

### DETAILED DESCRIPTION

Applicants believe that unstable nucleic acids within mutant cell nuclei cause or at least facilitate the further mutation of those cells. It is also felt that when dysfunctional cells, whose nuclei contain unstable nucleic acids, react with free amino ions or free ionic amino acids In an aqueous solution or an aqueous environment the resulting biosynthesis causes alignment and stabilization of those cells. Applicant asserts it is the process, at least in part, of the free amino ion and/or the free amino acid hydrogen bonding and/or ionicially bonding to the unstable nucleic acids in the nuclei within the mutant cells, promotes this alignment and resultant stabilization. One further beneficial result of this re-alignment is the unexpected result of those cells to return to normal function, meaning to function according to similar or analogous cells without the dysfunction. The biosynthesis occurs when a free amino ion and/or a free amino acid interacts ionically with the unstable nucleic acid resulting In stabilization and alignment. Applicant asserts this is why and how amniotic fluid keeps the proteins within fetal cell clusters stable. Specifically, there is present in amniotic fluid an overwhelming amount of free amino and/or free ionic amino acids causing constant biosynthesis and thereby cellular alignment. First process for preparing the argininoglycino-parabenzene-fulvate, or TAFA is through the production of an Intermediate reaction product useful in releasing free amino acid ions, which because of weak internal hydrogen bonding, allows for the release of amino ions. These ions provide numerous and various positive physiological effects In both humans and animals such as stabilizing unstable nucleic acids within dysfunctional cells by process of biosynthesis. This reaction product may also be used to boost and accelerate topical microcirculation and IS also useful In the manufacture of other reaction products. These additional reaction products allow for release of a greater quantity of free amino ions to facilitate positive physiological effects.

To obtain TAFA, those skilled In the art will dissolve at ultra high shear and at 80° Centigrade, 227.0 Grams of arginine (either d or I or a mixture thereof) in 1 liter of de-ionized water (USP). The ultra high shear mixing should take at least four minutes. Applicant uses a Ross homogenizer; however a Fischer PowerGen Model 1800D homogenizer would work satisfactorily. The mixing speed should be al least 4,000 rpm. The resulting solution should have pH of about 11.5. Reduce the mixing speed to 300 rpm and add 183.0 Grams of aminoacetic acid. Gradually increase mixing speed to 4000 rpm and mix at this speed for at least four minutes. pH should be about 9.2 but will rise to about 9.3 as solution cools, combine thereto at least a 1 molar equivalent of CHDFA, continue mixing for another 60 seconds and allow solution to stand. This a satisfactory end point for the TAFA complex and is ready for use in various topical, oral, nasal and internal applications.

To desiccate TAFA base solution to powdered form: Place the TAFA base solution in a Pyrex evaporating dish and then into vacuum oven. Set temperature to no more than 95° C. with a vacuum of between 1 and 1.5 negative atmospheres; evaporate until anhydrous. The resultant material will be crystalline. Remove from oven, cool and grind to a fine powder (4 microns or finer). The finished product will be a white powder.

### Experimental Results

Described below are a few examples and associated physical and biological responses to use of the present invention.

TAFA has exhibited vasodilatation qualities and may be used topically or internally to accelerate microcirculation without toxic side effects. This acceleration of microcirculation has many positive benefits. Wound Healing coupled with the restoration of cellular function, TAFA has exhibited the ability to aid and accelerate the healing of wounds, burns (of all degrees) and several types of chronic lesions. Amongst those lesions healed were radiation burns of the 3rd degree with complete suppression of scar formation), squamous cell carcinoma, basal cell carcinoma, psoriasis, venous stasis ulcers, decubitus ulcers (bedsores), deep surgical wounding (with suppression of scar formation) oral and genital herpes kidney issues, itching, and bleeding secondary to skin lisions.

### Hypertension

Encapsulated TAFA has demonstrated the ability to reduce hypertension without toxic side effects.

### Hair Restoration

The application of TAFA, which has been incorporated into a lotion has demonstrated the ability to restore follicular function (hair growth).

### Anti-Inflammatory

TAFA in all forms as shown the ability to reduce inflammation and control the discomfort resulting from inflammation.

### Pain Relief

TAFA is not an anesthetic, yet has demonstrated substantial pain relief, without diminishing topical sensation which is of great importance in treating wounds such as burns and skin lesions.

### Prostate

TAFA has demonstrated the ability to shrink a swollen prostate to normal size.

### Inhalation Product

To prepare a mixture for therapeutic inhalation via small volume nebulizer, use a 1:1 dilution of TAFA to 50% de-ionized water; combine 1 lite of the TAFA water mixture to 5 grams caffeine and 1 gm xylitol.

While there have been shown what are presently considered to be preferred embodiments of the present invention, it will be apparent to those skilled in the art that various changes and modifications can be made herein without departing from the scope and spirit of the invention. For example, persons skilled in the art may be enabled based on the disclosures herein to recognize additional techniques for producing the intermediate reaction products and/or the final reaction product described herein. Applications of the reaction products (in addition to the ones suggested herein) will undoubtedly become apparent to persons skilled in the art. However, the scope of the patent should be determined by the appended claims and their legal equivalence and not by the examples which have been given.

1. A composition consisting essentially of:
an arginine a glycine an alanine a para-amino benzoate and a carbohydrate derived fulvic acid graphically depicted according to structure VIII.

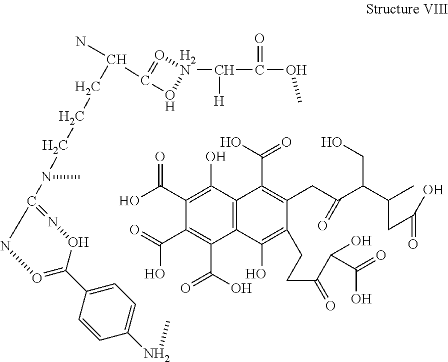

2. The composition according to claim 1 wherein there is between about 0.01 to about 20% molar excess PABA.

3. The composition according to claim 1 wherein there is between about 0.01 to about 80% molar excess CHDFA.

4. The composition according to claim 1 wherein there is between about 0.01 to about 20% molar excess alanine.

5. The composition according to claim 1 wherein there is between about 0.01 to about 20% molar excess arginine.

6. The composition according to claim 1 wherein at least one nitrogen is charged.

7. The composition according to claim 1 wherein at least one oxygen atom is charged.

8. The composition according to claim 1 wherein the compound further includes a metal.

9. The composition according to claim 8 wherein the metal is an ion.

10. The composition according to claim 8 wherein the metal is selected from the group Li, Na, Be, K, Ca, Sr, Ra, Cu, Zn and Mg.

11. The composition according to claim 8 wherein the metal is selected from period 4, 5 or 6 of the periodic table.

12. The composition according to claim 8 wherein the metal is chelated thereto.
